(19) United States
(12) Patent Application Publication
(10) Pub. No.: US 2025/0269161 A1
(43) Pub. Date: Aug. 28, 2025

(54) ANCHOR REGIONS FOR IMPLANTABLE MEDICAL DEVICE

(71) Applicants: W. L. Gore & Associates, Inc., Newark, DE (US); ViaCyte, Inc., San Diego, CA (US)

(72) Inventors: David Berg, Newark, DE (US); Karim Dudum, San Diego, CA (US); Evert Kroon, San Diego, CA (US); Laura Martinson, San Diego, CA (US); Craig McGreevy, San Diego, CA (US); Matthew McPherson, Newark, DE (US); Greg Rusch, Newark, DE (US); Benjamin A. Smith, Newark, DE (US); Jonathan Valdez, San Diego, CA (US); Janahan Arulmoli, San Diego, CA (US)

(21) Appl. No.: 19/115,902

(22) PCT Filed: Sep. 29, 2023
(86) PCT No.: PCT/US2023/075625

§ 371 Date: Mar. 27, 2025

Related U.S. Application Data
(60) Provisional application No. 63/412,204, filed on Sep. 30, 2022.

(51) Int. Cl. A61M 37/00

(52) U.S. Cl. CPC A61M 37/00

(57) ABSTRACT

Devices for encapsulating biological entities (e.g., cells) where the encapsulating devices are implanted into a tissue bed of a patient to provide biological therapy are disclosed. The encapsulation device includes a inactive region (e.g., weld area) located around the periphery of the device. The inactive region is non-porous and prevents cellular ingrowth and/or vascularization therein. An anchor region containing an ingrowth layer and a bonding layer may be attached or otherwise affixed to the inactive region. The open microstructure of the ingrowth layer permits for rapid cellular and/or vascular ingrowth to stabilize the encapsulation device within the host tissue. In some embodiments, inactive regions can be formed in location(s) on the cell retaining region of the encapsulation device.

### FIELD

The present disclosure relates generally to implantable medical devices, and more specifically, cell encapsulation devices that contain anchor region(s) on inactive region(s) where the anchor region(s) contain an ingrowth layer that permits rapid cellular and/or vascular ingrowth therein to stabilize the cell encapsulation device within host tissue.

### BACKGROUND

Biological therapies are increasingly viable methods for treating various diseases, such as diabetes, heart disease, Alzheimer's and Parkinson's diseases and various other diseases. In general, treatments such as cells, viruses, viral vectors, or other biological entities may be introduced into a patient by surgical methods. The biological entities may be first inserted into an encapsulation device that is then implanted into the patient at a target location. However, after implant, micromotion may occur which could destabilize the positioning of the device and elicit a chronic inflammatory response. In addition, micromotion may reduce collagenized ingrowth and vascularization required for proper function of the encapsulation device.

Thus, there remains a need for encapsulation devices with optimized stabilization (e.g., improved host tissue integration and anchoring of the encapsulation device) and function of the encapsulation device within the body.

### SUMMARY

In one Aspect (“Aspect 1”), a cell encapsulation device includes a cell retaining region, an inactive region, and an anchor region disposed onto at least a portion of the inactive region where the anchor region contains an open microstructure configured to promote tissue ingrowth.

According to another Aspect (“Aspect 2”) further to Aspect 1, where the open microstructure comprises, a porous polymer having a plurality of anchoring features defined by a dimension between about 0.1 microns and about 100 microns.

According to another Aspect (“Aspect 3”) further to Aspect 1 or Aspect 2, where the inactive region is arranged along a perimeter of the cell retaining region.

According to another Aspect (“Aspect 4”) further to any one of the previous Aspects, including at least one inactive region positioned on the cell retaining region.

According to another Aspect (“Aspect 5”) further to any one of the previous Aspects, where the anchor region comprises expanded polytetrafluoroethylene.

According to another Aspect (“Aspect 6”) further to any one of the previous Aspects, where at least 15% of an area of the inactive region is covered by the anchor region.

According to another Aspect (“Aspect 7”) further to any one of the previous Aspects, where an anchor region comprises a bonding layer and an ingrowth layer.

According to another Aspect (“Aspect 8”) further to any one of the previous Aspects, where the bonding layer comprises a first plurality of fibrils and the ingrowth layer comprises a second plurality of fibrils and the bonding layer has a fibril density that is greater than a fibril density of the ingrowth layer.

According to another Aspect (“Aspect 9”) further to any one of the previous Aspects, where the ingrowth layer is configured for permitting tissue ingrowth within the pores of the ingrowth layer.

According to another Aspect (“Aspect 10”) further to any one of the previous Aspects, where wherein a ratio of active region/inactive region is from 50% to 150%

In one Aspect (“Aspect 11”), a cell encapsulation device includes an active area covering at least one reservoir containing cells and defined by a perimeter and a surface area, a seal region arranged around the perimeter of the active region, an open microstructure layer disposed onto at least a portion of the anchor region, where the open microstructure is defined by interconnected fibrils, and where the open microstructure is defined by a thickness of between 5 microns and 600 microns.

According to another Aspect (“Aspect 12”) further to Aspect 11, where the fibrils of the open microstructure have a dimension of between 0.1 microns and 100 microns.

According to another Aspect (“Aspect 13”) further to Aspect 11 or Aspect 12, where the fibrils of the open microstructure have a dimension of less than 1 micron.

According to another Aspect (“Aspect 14”) further to any one of Aspects 11 to 13, where at least 15% of the seal region is covered by the anchor region.

According to another Aspect (“Aspect 15”) further to any one of Aspects 11 to 14, where between 40% and 95% of the seal region is covered by the anchor region.

According to another Aspect (“Aspect 16”) further to any one of Aspects 11 to 15, where wherein between 60% and 99% of the seal region is covered by the anchor region.

According to another Aspect (“Aspect 17”) further to any one of Aspects 11 to 16, where the open microstructure is welded with the seal region.

According to another Aspect (“Aspect 18”) further to any one of Aspects 11 to 17, where the open microstructure is composed of a first layer and a second layer, wherein each of the first layer and the second layer comprise fibrils, and wherein the first layer is defined by a higher fibril density than a fibril density of the second layer.

In one Aspect (“Aspect 19”), an anchor region for use with an implantable medical device configured for facilitating tissue integration includes a bonding layer and an ingrowth layer attached to the bonding layer, where the ingrowth layer comprises a porous polymer having a plurality of anchoring features defined by a dimension between about 0.1 microns and about 100 microns, and where the porous polymer is configured for integration with tissue.

According to another Aspect (“Aspect 20”) further to Aspect 19, where the bonding layer is configured for welding with the implantable medical device.

According to another Aspect (“Aspect 21”) further to Aspect 19 or Aspect 20, where the bonding layer has a first plurality of fibrils, the ingrowth layer has a second plurality of fibrils and where a fibril density of the bonding layer is greater than a fibril density of the ingrowth layer.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a top schematic view of a cell encapsulation device, according to some embodiments;

FIG. 2 is a cross sectional schematic view of the cell encapsulation device of FIG. 1 in accordance with some embodiments;

FIG. 2A illustrates a cross sectional schematic view of a semipermeable membrane in accordance with some embodiments;

FIG. 3 is a top schematic view of a cell encapsulation device in accordance with some embodiments;

FIG. 4 is a cross sectional schematic view of the cell encapsulation device of FIG. 3 in accordance with some embodiments;

FIG. 5 is a schematic top view of a cell encapsulation device having an open anchoring region on a perimeter of the device in accordance with some embodiments;

FIG. 6 is a schematic illustration of a photograph of an anchor region containing an ingrowth layer and a portion of the active region (e.g., mesh) of a cell encapsulation device in accordance with some embodiments;

FIG. 7 is top-down view of the most open expanded polytetrafluoroethylene (ePTFE) layer of Example 1 taken with optical microscope at 50× magnification in accordance with some embodiments;

FIG. 8 is a scanning electron micrograph (SEM) of a cross section of the biocompatible membrane composite formed by the Example 1 in accordance with some embodiments;

FIGS. 9A-9E are representative histology images demonstrating improved collagenized tissue ingrowth in Examples 1 and 3 and no improvement to collagenized tissue ingrowth in the active area of Comparative Examples 4, 5, F and 6 in accordance with some embodiments; and

FIGS. 10A-10E are representative histology images of capsule thicknesses of Examples 1 and 3 as well as Comparative Examples 4, 5, and 6 in accordance with some embodiments.

### DETAILED DESCRIPTION

Persons skilled in the art will readily appreciate that various aspects of the present disclosure can be realized by any number of methods and apparatus configured to perform the intended functions. It should also be noted that the accompanying figures referred to herein are not necessarily drawn to scale and may be exaggerated to illustrate various aspects of the present disclosure, and in that regard, the figures should not be construed as limiting. Directional references such as “up,” “down,” “top,” “left,” “right,” “front,” and “back,” among others are intended to refer to the orientation as illustrated and described in the figure (or figures) to which the components and directions are referencing. It is to be noted that all ranges described herein are exemplary in nature and include any and all values in between. In addition, all references cited herein are incorporated by reference in their entireties. The terms “implantable medical device” or “implantable device” may be used interchangeably with the term “cell encapsulation device” or “encapsulation device” herein.

The present disclosure is directed to devices for encapsulating biological entities (e.g., cells), where the encapsulating devices are implanted into a patient, such as into a tissue bed, to provide biological therapy. The encapsulation device includes a inactive region located around the periphery of the device. The inactive region is non-porous and prevents cellular ingrowth and/or vascularization therein. An anchor region containing an ingrowth layer and a bonding layer may be attached or otherwise affixed to the inactive region. The open microstructure of the ingrowth layer permits for rapid cellular and/or vascular ingrowth to stabilize the encapsulation device within the host tissue. In some embodiments, inactive regions can be formed in location(s) on the cell retaining region of the encapsulation device. It is to be appreciated that the term “about” as used herein denotes +/−10% of the designated unit of measure.

Biological entities suitable for encapsulation and implantation using the devices described herein include cells, viruses, viral vectors, bacteria, proteins, antibodies, and other bioactive entities. For simplicity, herein the biological entity is referred to as a cell, but nothing in this description limits the biological entity to cells or to any particular type of cell, and the following description applies also to biological entities that are not cells. Various types of prokaryotic cells, eukaryotic cells, mammalian cells, non-mammalian cells, and/or stem cells may be used with the cell encapsulation devices of the present invention. In some embodiments, the cells are microencapsulated within a biomaterial of natural or synthetic origin, including, but not limited to, a hydrogel biomaterial. In some embodiments, the cells secrete a therapeutically useful substance. Such substances include hormones, growth factors, trophic factors, neurotransmitters, lymphokines, antibodies, or other cell products which provide a therapeutic benefit to the device recipient. Examples of such therapeutic cell products include, but are not limited to, insulin, growth factors, interleukins, parathyroid hormone, erythropoietin, transferrin, and Factor VIII. Non-limiting examples of suitable growth factors include vascular endothelial growth factor, platelet-derived growth factor, platelet-activating factor, transforming growth factors, bone morphogenetic protein, activin, inhibin, fibroblast growth factors, granulocyte-colony stimulating factor, granulocyte-macrophage colony stimulating factor, glial cell line-derived neurotrophic factor, growth differentiation factor-9, epidermal growth factor, and combinations thereof. It is to be appreciated that throughout this disclosure the terms “cell” or “cells” could be replaced by “biological entity” or “biological entities”, respectively. In addition, the terms “cell encapsulation device”, “encapsulation device”, and “device” may be used interchangeably herein.

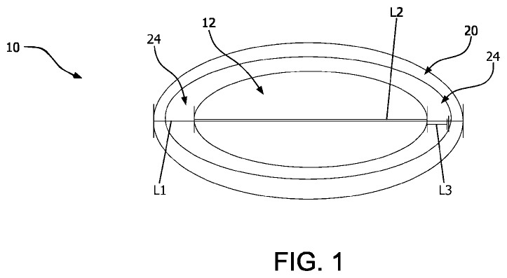

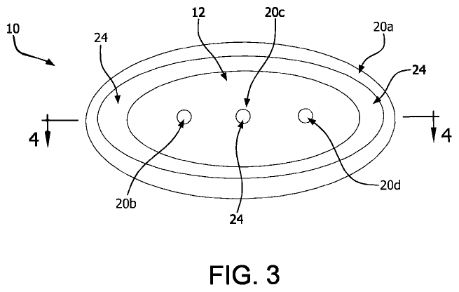

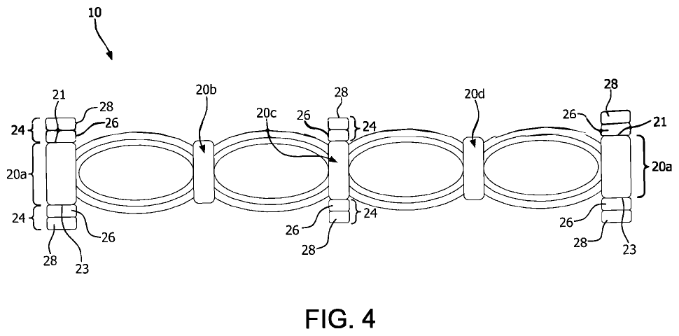

FIG. 1 depicts a top view of an embodiment of a cell encapsulation device 10. It is to be appreciated, however, that embodiments described herein may be applied to a wide variety of implantable medical devices and a cell encapsulation device such as is illustrated in FIG. 1 is meant only to be exemplary in nature. It is to be appreciated that any device that is meant to reside or resides within a patient's body is considered to be within the purview of this disclosure. As shown in FIG. 1, the cell encapsulation device 10 has a cell retaining region 12 (e.g., reservoir), an inactive region 20, and an anchor region 24 disposed onto the inactive region 20. The inactive region 20 is covered by the anchor region 24, including, for example, the inactive region 20 overlapping, coextensive with, overlaid by, or otherwise covered or partly covered by the anchor region 24. In some embodiments, the anchor region 24 may be disposed only onto a portion of the inactive region 20. In further embodiments, the anchor region 24 may also and/or alternatively be positioned on other regions of the device 10 that contain inactive areas. As used herein, the term “inactive areas” defines areas or regions of the cell encapsulation device (or implantable medical device) that are not directly above cell containing region(s) 12 of the cell encapsulation device 10 and/or an area(s) in which no cellular or vascular ingrowth may occur, and areas that cannot provide mass transport into or out of a cell containing region(s) 12. The term “active areas” as used herein is meant to denote areas or regions that are directly above the cell containing region(s) 12 and/or areas or regions that are capable of mass transport in and out of the cell retaining region(s) 12. As shown in FIG. 1, the cell encapsulation device 10 may have two (2) anchor regions 24 disposed thereon. As will be described further herein with reference to FIGS. 3 and 4, in some embodiments, the cell encapsulation device 10 has two (2) or more anchor regions 24 disposed thereon.

The inactive region 20 may be defined as the area within which the two composite layers are welded, or otherwise adhered and/or joined together to form a seal around the periphery of the device 10. As shown in FIG. 1, the inactive region 20 is defined by an area around the outer periphery of the cell encapsulation device 10, although the inactive region 20 may take on a variety of configurations. In the embodiment depicted in FIG. 1, the inactive region 20 defines the outer perimeter of the cell encapsulation device 10 and has an overall length L1. As illustrated in FIG. 1, the overall length LI defines the greatest dimension of the cell encapsulation device 10. The cell encapsulation device 10 is also defined by a length L2, which is the greatest dimension of the cell retaining region 12. Further, a length L3 is defined by the difference between the overall length L1 of the cell encapsulation device 10 and length L2, the greatest length of the cell retaining region 12. While the above dimensions are referenced as lengths, in some embodiments (not depicted), the cell retaining region 12 may have a different configuration and the greatest dimension may be defined as a width and/or a thickness.

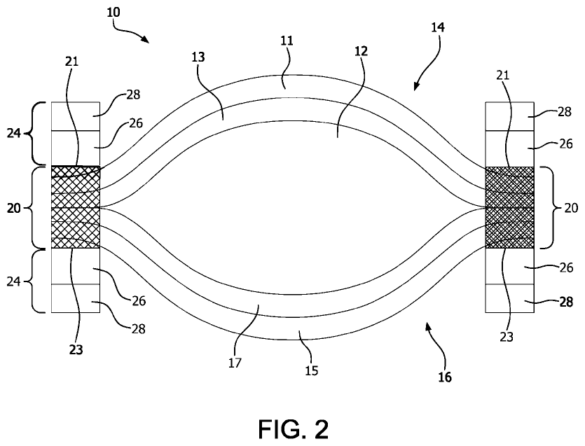

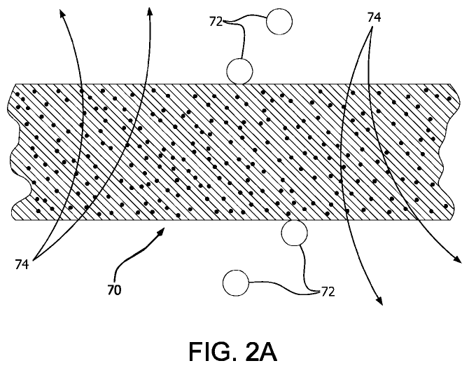

As illustrated in the cross-sectional view of FIG. 2, the cell encapsulation device 10 may include a first composite layer 14 and a second composite layer 16 that are sealed around their peripheries to form the cell retaining region 12. The cell retaining region 12 is configured to receive cells or other therapeutic biological entities. The first composite layer 14 and the second composite layer 16 each include an outer porous layer 11, 15 disposed adjacent to the inner porous layer 13, 17, respectively. The inner porous layers 13, 17 of the first and second composite layers 14, 16 are impervious to cell ingrowth. For example, both inner porous layers 13, 17 have an average pore size that is sufficiently small so as to prevent vascular ingrowth (e.g., less than about 1 micron as measured by porometry). In contrast, the outer porous layers 11, 15 are sufficiently porous to permit the growth of tissue into the pores of the outer porous layer (e.g., greater than about 1 micron as measured by porometry). Ingrowth of tissue through the outer porous layers 11, 15 facilitates nutrient and bio-molecule transfer from the body to the cells encapsulated in the cell retaining region 12 of the device 10. In some embodiments, the cell retaining region 12 may be formed of one or more single layers (e.g., layers that are not composite layers) (not shown). In further embodiments, the cell retaining region 12 may be formed solely of a cell impermeable layer (not shown) or a cell impermeable layer and two (or more) cell permeable layers (not shown). Areas that are defined by the outer porous layers 11, 15 that are capable of mass transport in and out of the cell retaining region 12 may be referred to herein as active areas. Implantable devices that contain more than first composite layer 14 and/or second composite layer 16 are considered to be within the purview of the invention.

The first composite layer 14 and the second composite layer 16 may be joined together around the perimeter of the first and second composite layers 14, 16 to form an inactive region 20. The inactive region 20 forms an outer periphery of the cell encapsulation device 10. The first composite layer 14 and the second composite layer 16 may be bonded together through any variety of welding techniques, fusing mechanisms, or adhering mechanisms. In some embodiments, the first and second composite layers 14, 16 are joined via thermoplastic welding, ultrasonic welding, fusing, adhesives, mechanical engagement between the layers, and various other applicable methods of bonding polymer layers as would be known to one of skill in the art.

In some embodiments, one or both of the first composite layer 14 and the second composite layer 16 of the cell encapsulation device 10 is made, primarily or entirely, of a semipermeable material having selective sieving and/or porous properties. The semipermeable material controls the passage of solutes, biochemical substances, viruses, and cells, for example, through the material, primarily on the basis of size. In embodiments where the semipermeable material is porous only through a portion of its thickness, the molecular weight cutoff, or sieving property, of the semipermeable membrane begins at the surface. As a result, certain solutes and/or cells do not enter and pass through the porous spaces of the material from one side to the other. FIG. 2A depicts a cross-sectional view of a porous material 70 useful in cell encapsulation devices 10 described herein, where the selective permeability of the porous material 70 excludes cells 72 from migrating or growing into the spaces of the porous material 70 while permitting bi-directional flux of solutes 74 across the thickness of the porous material 70. The areas that are defined by the semipermeable membrane(s) that are capable of mass transport in and out of the cell retaining region 12, may also be referred to herein as active areas.

The process of joining the first and second composite layers 14, 16 may cause the exterior surfaces of the inactive region 20 to be non-porous. In some embodiments, the first and second composite layers 14, 16 may be fused together, such as with heat and pressure, without the use of an adhesive. Fusing with heat and pressure may cause a loss of surface porosity and may result in the densification of any microstructure at the surface where the fusion occurred. Thus, after fusing (with no adhesive) the first and second composite layers 14, 16, a non-porous surface is formed on the exterior of the inactive region 20. In some embodiments, thermoplastic welding may be used, which causes pores of the first and second composite layers 14,16 to be filled, partially filled, covered, embedded, or otherwise imbibed with molten polymer material. In an additional embodiment where the first and second composite layers 14, 16 are self-adhering, joining the first and second composite layers 14, 16 through methods such as thermal or ultrasonic welding may cause the pores to collapse and become smooth and non-porous. The bonding of the first and second composite layers 14, 16 results in an area where the transfer of nutrients or therapies in and/or out of the inactive region 20 is prohibited. Further, any cellular or tissue integration into the inactive region 20 is unable to occur. As such, the inactive region 20 may be an inactive area of the cell encapsulation device 10.

The cell encapsulation device 10 may include additional inactive regions that are separate from the seal area 20. One non-limiting example of an inactive region may be a structural frame or stiffening member that is positioned around the perimeter of the device 10 to provide rigidity and handleability to the encapsulation device 10. In some embodiments, additional elements such as a fill tube that is used for delivering therapeutic biological entities into the device 10 may be an inactive region of the device 10, as it does not permit cellular or tissue ingrowth. Various other inactive areas may be defined on the device 10 such as, but not limited to, a structural frame, suture tabs, or holes.

The material forming the frame is not particularly limited so long as provides the necessary stiffness and is compatible with the implant environment and having the necessary stiffness. Non-limiting examples of useful materials include, but are not limited to, polymer materials such as polyetheretherketone (PEEK), polyethylene terephthalate (PET), polypropylene, polyethylene, polymethyl methacrylate, polyethyl methacrylate, polyacrylate, poly-alpha-hydroxy acids, polycaprolactones, polydioxanones, polyesters, polyglycolic acid, polyglycols, polylactides, polyorthoesters, polyphosphates, polyoxaesters, polyphosphoesters, polyphosphonates, polysaccharides, polytyrosine carbonates, silicones, polyurethanes, polyurethanes with ionic or mesogenic components made by a pre-polymer method, a block copolymer of polyethylene terephthalate (PET) and polyethyleneoxide (PEO), block copolymers containing polystyrene and poly(1,4-butadiene), and an ABA triblock copolymer made from poly(2-methyl-2-oxazoline) and polytetrahydrofuran, and copolymers or polymer blends thereof. Metallic frames can also be incorporated using materials such as spring tempered 316 SST; a spring-tempered cobalt-chromium alloy, such as Co-28Cr-6Mo or Co-35Ni-20Cr-10Mo; a spring-tempered titanium-based alloy, such as Ti-6Al-4V or a spring-tempered nickel-titanium alloy, such as Nitinol or copper-aluminum-nickel, copper-zinc-aluminium, aluminum, and iron-manganese-silicon alloys. The frame materials may be a material that is inherently biocompatible or may be a material that lacks inherent biocompatibility but is rendered biocompatible, such as with a biocompatible coating. Non-limiting examples of inherently biocompatible frame materials include PEEK, Nitinol or Ti-6A1-4V.

As shown in FIG. 2, an anchor region 24 may be disposed onto at least a portion of the inactive region 20 to provide a porous outwardly facing surface that enables tissue integration and ingrowth. In other words, and as will be described further herein, the inactive region 20 has no propensity for tissue integration or ingrowth but the anchor region 24 has a propensity for tissue integration such that tissue integration is enabled into the anchor region 24.

The anchor region 24 may be disposed onto at least a portion of the surface of the inactive region 20. In some embodiments, the anchor region 24 may be disposed onto the inactive region 20, for example fused, welded, adhered, or otherwise attached to the inactive region 20. In some embodiments, the surface of the inactive region 20 may include an upper surface 21 and a lower surface 23. The anchor region 24 may be attached to at least a portion of one or both of the upper surface 21 and the lower surface 23 of the inactive region 20.

In some embodiments, the anchor region 24 has a surface area that is about 90% of a surface area of the inactive region 20. In these embodiments, the surface area of the inactive region 20 may include the total surface area of the inactive region 20 on both the upper surface 21 and the lower surface 23 of the inactive region 20. As such, in the embodiments where the anchor region 24 is disposed on both the upper surface 21 and the lower surface 23 of the inactive region 20, the surface area of the anchor region 24 may be defined as the total surface area of the anchor region 24 disposed on both the upper surface 21 and the lower surface 23.

In some embodiments, the value of the surface area of the anchor region 24 may vary. For example, the anchor region 24 may have a surface area that is at least 15% of the surface area of the inactive region 20. In further embodiments, the anchor region 24 may have a surface area that is at least 15% of a total surface area of the inactive region 20. In other non-limiting examples, the anchor region 24 may have a surface area between about 5% and about 100% of the surface area of the inactive region 20, a surface area between about 5% and about 99% of the surface area of the inactive region 20, a surface area between about 5% and about 90% of the surface area of the inactive region 20, a surface area between about 15% and about 85% of the surface area of the inactive region 20, a surface area between about 20% and about 75% of the surface area of the inactive region 20, a surface area between about 30% and about 60% of the surface area of the inactive region 20, or a surface area between about 40% and about 50% of the surface area of the inactive region 20. In further embodiments, the anchor region 24 may have a surface area between about 15% to about 100%, between about 15% and about 99%, between about 25% and about 99%, between about 35% and about 99%, between about 45% and about 99%, between about 55% and about 99%, between about 65% and about 99%, or between about 75% and about 99% of the surface area of the inactive region 20.

The anchor region 24 may be defined as a portion on the inactive region 20 that has an open microstructure disposed thereon and is exposed directly to the native tissue (i.e., host tissue) when implanted. As shown in FIG. 2, the anchor region 24 may be formed of an ingrowth layer 28 and a bonding layer 26. The ingrowth layer 28 of the anchor region 24 has an open microstructure that may promote ingrowth of cells and tissue ingrowth onto and within the anchor region 24. In some embodiments, the open microstructure is a node and fibril microstructure. As used herein, the term “open” is meant to denote that the region or layer being described is cell permeable and cells may enter and/or exit the layer or region to allow for cellular ingrowth and/or tissue ingrowth/integration. It is to be appreciated that the bonding layer 26 does not have sufficient porosity to permit cellular or vascular ingrowth. Herein, layers that restrict or prevent tissue ingrowth and/or the integration of weld material may be referred to as “tight” layers.

The open microstructure of the ingrowth layer 28 may include a plurality of anchoring features that may permit cellular and/or tissue ingrowth onto and into the outer porous layers 11, 15, of the cell encapsulation device 10 to wrap around and anchor within the ingrowth layer 28 of the anchor region 24. The anchoring features may be fibrils and/or fibers. In addition, the anchoring features may be defined by a dimension, such as a diameter, that has a value between about 0.1 microns and about 100 microns. In some embodiments, the size of the anchoring features may range between about 0.1 microns and about 80 microns, between about 0.1 microns and about 75 microns, between about 0.1 microns and about 70 microns, between about 0.1 microns and 65 microns, between about 0.1 microns and 50 microns, between about 0.1 and 25 microns, between about 0.1 microns and about 10 microns, between about 0.1 microns and about 2 microns, or between about 0.1 microns and 0.5 microns. In some embodiments, the dimension of the anchoring features may be defined as the average dimension, or diameter, of the anchoring features of the open microstructure. In these examples, the dimension of the anchoring features may be measured through scanning electron micrograph (SEM) images taken of the anchoring features.

In some embodiments, the rate at which tissue ingrowth penetrates and encapsulates the anchoring features in the ingrowth layer 28, and thus the anchor regions 24, to anchor the encapsulation device 10 within the surrounding tissue ingrowth is faster than tissue integration in other areas of the cell encapsulation device 10. The faster integration of tissue ingrowth into the ingrowth layer 28 stabilizes the cell encapsulation device 10 in the tissue bed and reduces the effect of any micromotion or shifting of the encapsulation device 10. Once tissue has integrated into the anchor regions 24, the device 10 may be stabilized within the host tissue (i.e., micromovements/micromotions of the device 10 are reduced), which, in turn, facilitates the development of vasculature around the encapsulation device 10 and into the outer porous membranes 11, 15. As a result, nutrients may be delivered to the cell retaining region 12 more quickly.

As discussed above, the anchor region 24 may include a bonding layer 26 that is configured for direct coupling to the inactive region 20 and an ingrowth layer 28 that is configured for coupling to the bonding layer 26 and for exposure to the native tissue and subsequent ingrowth for the purpose of anchoring the cell encapsulation device 10. In some embodiments, the density of the fibers or fibrils of the bonding layer 26 may be greater than the density of the fibers or fibrils of the ingrowth layer 28 of the anchor region 24 as determined by pore size (e.g., bonding layer 26 pore size less than 1 micron). In this way, during a thermoplastic welding process of the anchor region 24 onto the inactive region 20, the molten polymer (e.g., weld material) may enter the bonding layer 26 but is inhibited from flowing or traveling into the ingrowth layer 28, thereby maintaining the ability of the ingrowth layer 28 for propagation of cellular and/or tissue ingrowth into the anchor region 24.

In some embodiments, the bonding layer 26 may be a component of the inactive region 20, rather than a component of the anchor region 24. For instance, the inactive region 20 may include the bonding layer 26 prior to the attachment of the anchor region 24 onto the inactive region 20. In other embodiments, the ingrowth layer 28 may be a continuation of the bonding layer 26 so as to provide an open layer that native tissue (i.e., host tissue) can grow into and anchor the cell encapsulation device 10. In some embodiments, the bonding layer 26 may be modified such that it is capable of cellular and/or tissue integration and for bonding with the inactive region 20. In this way, a top portion of the bonding layer 26 may have a more open structure (e.g., a lesser fibril density) than a bottom portion of the bonding layer 26. In some embodiments, the bonding layer 26 may be a tight membrane (e.g., an expanded polytetrafluoroethylene membrane (ePTFE)) or other microporous membrane.

In some embodiments, the anchor region 24 (and/or ingrowth layer 28) may include any number of polymer layers. For example, the anchor region 24 may include two layers (e.g., a bilayer membrane), three layers (e.g., a tri-layer membrane), four layers, five layers, or even more. When a multi-layer anchor region 24 is utilized, one side of a multi-layer polymer membrane is configured for direct contact and coupling with the inactive region 20 while the opposing side of the multi-layer polymer membrane is configured for exposure to native tissue and subsequent cell and/or tissue ingrowth for the purpose of anchoring the encapsulation device 10. The bonding layer 26 may be a tight layer or an open layer. When the bonding layer 26 is an open layer, another layer may be present between the bonding layer 26 and the ingrowth layer 28 that prevents adhesive and/or weld material penetration from the bonding layer 26 into the ingrowth layer 28. Additionally, the bonding layer 26 may be a composite layer with an open layer to allow thermoplastic polymer to melt into and a tight layer to prevent the thermoplastic polymer from occluding the ingrowth layer 28. The multi-layer polymer membrane can take numerous configurations, such as, but not limited to, an open-tight-open structure, a tight-open structure, or a tight-open-open structure.

In some embodiments, the anchor region 24 may consist of a single expanded polytetrafluoroethylene layer (ePTFE layer), a bilayer containing an ePTFE layer where one of the layers has a first pore size and the second layer has a second pore size, and the first pore size is different from the second pore size. For example, a first pore size could be less than about 1 micron and a second pore size could be greater than about 2 microns. A tri-layer containing an ePTFE layer may include a large pore size on the outer layers and a smaller pore size on the inner layer. In a tri-layer composite containing ePTFE, the ePTFE composite may have a tight/medium/open pore size in progression. Non-limiting layers that may be included in the anchor region 24 include a non-woven layer (such as spunbonded non-woven polyethylene terephthalate (PET), a bioabsorbable non-woven, polyether ether ketone (PEEK), a non-woven laminated to an ePTFE tight pore membrane, an electrospun membrane, a polytetrafluoroethylene (PTFE) electrospun membrane, and a porous membrane such as formed by dissolving a salt incorporated into the membrane (i.e., salt leeching). Methods of making porous membranes include solvent induced phase separation, vapor induced phase separation, track etching, and sintering.

As illustrated best in FIG. 2, the ingrowth region 28 has a thickness. In some embodiments, the thickness may be between about 5 microns and about 600 microns. In some embodiments, the thickness may range between about 10 microns and 600 microns, between about 15 microns and about 600 microns, between about 20 microns and about 600 microns, between about 20 microns and about 400 microns, between about 20 microns and about 100 microns, or between about 20 microns and about 50 microns. The ingrowth region 28 may have the same thickness on each side of the inactive region 20. However, in other embodiments, the ingrowth region 28 may be designed such that the thickness is greater on one side of the cell encapsulation device 10 than on the opposing side of the device 10.

While the anchor region 24 is described herein as being disposed onto the inactive region 20, in other embodiments, the anchor region 24 may be disposed onto any inactive area(s) of the cell encapsulation device 10, as will be described further with reference to FIGS. 3 and 4. For example, in embodiments where a first membrane layer forms the first composite layer 14 and a second membrane layer forms the second composite layer 16, the cell retaining region 12 may be designed such that portions of the first membrane layer and the second membrane layer may be sealed together in portions of the cell retaining region 12 such that the cell retaining region 12 has several compartments or reservoirs therein. Inactive regions 20 may also be formed where portion(s) of the first composite layer 14 and second composite layer 16 are sealed to each other. The sealed areas between the first membrane layer and the second membrane layer or the first composite layer 14 and the second composite layer 16 may then define inactive regions 20 that an anchor region 24 could be bonded thereto. Various other inactive regions 20 include a frame or stiffening member positioned around the perimeter of the device 10 or around active areas.

Once the cell encapsulation device 10 is implanted into a subject, tissue may grow into engagement with the microstructure of the ingrowth layer 28 of the anchor region 24 and propagate into the open microstructure. Such tissue ingrowth allows the tissue located around the cell encapsulation device 10 to secure to the cell encapsulation device 10 and/or anchor the positioning of the cell encapsulation device 10 within the target location. In particular, native tissue may engage with the cell encapsulation device 10 around the entirety of the device 10 to inhibit any micromotion or shifting of the cell encapsulation device 10 within the tissue bed. As previously described herein, the rapid tissue integration in the anchor regions 24 provides for quicker stabilization of the cell encapsulation device 10 within the patient which, in turn, allows for vasculature more quickly around the encapsulation device 10. Nutrients may then be transferred into the cell retaining region 12. As a result, the targeted therapy of the encapsulation device 10 may be more efficiently established to optimize the function of the cell encapsulation device 10.

The cell encapsulation device 10 may include more than one inactive region 20 and/or anchor region 24. For example, FIG. 3 illustrates an additional embodiment of the cell encapsulation device 10 which includes several inactive areas formed on the cell retaining region 12 of the encapsulation device 10. FIG. 3 is a top schematic view of the cell encapsulation device 10 having four inactive regions (i.e., 20a, 20b, 20c, 20d). The cell encapsulation device 10 includes a first inactive region 20a which may be the same as the inactive region 20 described above with respect to FIG. 2. As shown in FIG. 3, the cell encapsulation device 10 further includes a second inactive region 20b, a third inactive region 20c, and a fourth inactive region 20d. The second, third, and fourth inactive regions 20b-d, respectively, may be circular inactive regions disposed over the cell retaining region 12. It is to be appreciated that the circular inactive regions shown in FIG. 3 are exemplary in nature, and any geometric shape such as a triangle, square, oval, and the like may form an inactive region 20. In some embodiments, the inactive regions 20b-d are point bonded inactive regions. In addition, the inactive regions 20a-20d are inactive regions formed on the cell retaining region 12 of the encapsulation device 10.

FIG. 4 is a cross-sectional view of the cell encapsulation device 10 of FIG. 3 taken along line 4-4. As illustrated, the inactive region 20a, which extends around a perimeter of the cell encapsulation device 10, comprises an anchor region 24 disposed thereon. Further, inactive region 20c also comprises anchor regions 24 disposed on both sides of the inactive region 20a. In the depicted embodiment, both the inactive region 20a and the inactive region 20c include anchor regions 24 on both sides of the inactive regions 20a, 20c to allow tissue integration within the anchor regions 20a and 20c. While inactive regions 20b, 20d are not illustrated as having an anchor region 24 disposed thereon, in some embodiments, the anchor region 24 may be positioned on one or both of the inactive regions 20b, 20d. It is to be noted that the anchor regions 24 positioned on inactive region 20c each contain a bonding layer 26 and an ingrowth layer 28. In addition, the cell encapsulation device 10 may include any number of inactive regions 20, for example, one, two, three, four, or five or more inactive regions 20. One, some, or all of these inactive regions 20 may be configured to have anchor regions 24 welded, adhered or otherwise attached thereon. An increased amount of ingrowth region 24 on the inactive regions 20 increases the area of the cell encapsulation device 10 that is available for rapid tissue ingrowth.

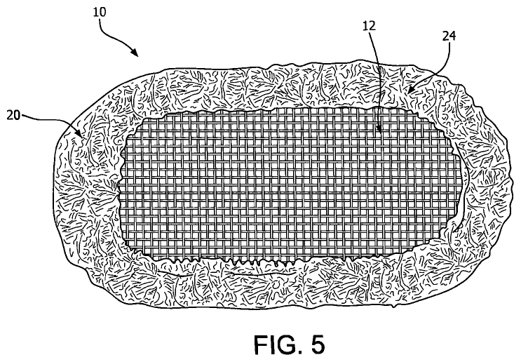

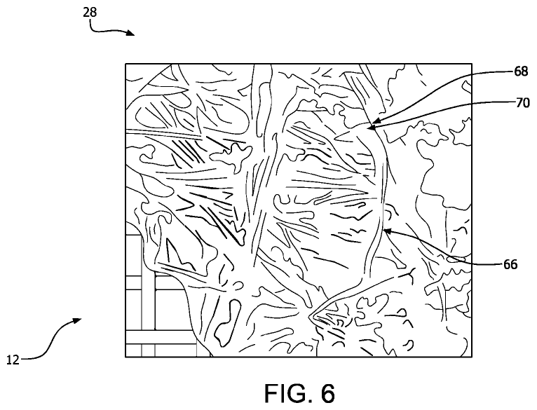

FIG. 5 illustrates a top view of the cell encapsulation device 10 of FIG. 1 having a modified anchor region 24 with tissue ingrowth layer disposed thereon. The active area 12 is shown for illustrative purposes only. FIG. 6 illustrates an enlarged view of a portion of the anchor region 24 of FIG. 5. The active area 12 is also depicted in the in FIG. 5. With reference to FIG. 6, the ingrowth layer 28 includes anchoring features that may include a plurality of fibrils 66 which terminate or originate at a node 68. Voids between the nodes 68 and fibrils 66 are defined as pores 70.

While the above-described embodiments reference the anchor region 24 as being disposed on the inactive region 20, the anchor region 24 may be disposed onto any non-porous area, or otherwise inactive area, that is desired to be covered with an ingrowth layer 28. The above-described embodiments of the anchor region 24 in combination with the device 10 may be especially useful in instances where the inactive area represents a significant portion of the device 10. For example, if the area of the inactive region is about equal to or greater than the area of the active regions, for example the area of the cell retaining region 12, then there may be an increased difficulty in achieving rapid anchoring of the device 10 within a subject. This becomes more pronounced when the area of the active region is less than the area of the inactive region, for example 90% active area/inactive area, this anchoring in the inactive areas becomes more desirable and even more at a ratio of 80% active area/inactive area. As such, it may be desirable to increase the surface area that is capable for interfacing and promoting tissue ingrowth on the tissue facing surface of the device 10. In some embodiments, the ratio of active region/inactive region may be from about 50% to about 150%, from about 50% to about 125%, from about 50% to about 100%, or from about 50% to about 75%.

Further, while the implantable device is largely referred to herein as a cell encapsulation device 10, the anchor region 24 may be applied to any variety of implantable medical devices. The anchor region 24 may be disposed onto any inactive (e.g., non-porous) surface of any device that may be designed for insertion into a patient. In this way, the advantages of the anchor region 24 may be used in combination with various types of devices that need an increased stability in the patient.

### TEST METHODS

### In Vivo New Zealand White Rabbits Study to Evaluate Host Tissue

Sterilized, empty encapsulation devices (i.e., no cells) were sealed at the fill tube prior to sterilization and implanted subcutaneously in the dorsum of New Zealand white rabbits using a blunt dissection delivery technique. After approximately 7 and 30 days, the animals were euthanized, and devices were retrieved for histological imaging.

The tissue samples were processed such that the skin and subcutaneous tissue were reflected to expose the implanted encapsulation devices. The devices were identified using digital radiography (Faxitron UltraFocus System) when needed prior to removing the encapsulation device and surrounding tissue en bloc. Device orientation was marked with staples. All explanted devices and surrounding tissue were immersed in 10% neutral buffered formalin. Each device specimen was assigned a unique accession number.

Three cross-sections were taken from each specimen. The three sections from each device were embedded together in paraffin, cut into 5-10 microns thick sections, placed on a slide and stained with hematoxylin and eosin (H&E) and Masson's Trichrome.

Images of the slide were captured using a Nikon DS-Fi Series camera and Nikon NIS Elements Microscope Imaging software. At least three magnification images of each slide were captured. Measurements were taken using the Nikon NIS Elements Microscope Imaging software which is calibrated using a certified microscope micrometer.

### SEM Sample Preparation and Thickness Measurement

SEM samples were prepared by first fixing the membrane composite or membrane composite layer(s) to an adhesive for handling, with the side opposite the side intended for imaging facing the adhesive. The film was then cut to provide an approximately 3 mm×3 mm area for imaging. The sample was then sputter coated using an Emitech K550X sputter coater and platinum target. Images were then taken using a FEI Quanta 400 scanning electron microscope from Thermo Scientific at a magnification and resolution that allowed visualization of a sufficient number of features for robust analysis while ensuring each analyzed feature's minimum dimension was at least five pixels in length. Layer thickness was measured via cross-sectional SEM images.

### Surface Roughness

Surface roughness was measured using a Keyence VK-X1000 Laser Scanning Confocal Microscope and associated Multi File Analyzer Software. Samples were immobilized to a microscope stage with tape in a peripheral region of the device being measured. Images were then taken using the VK-X1000 at a magnification of 20×. A surface area of approximately 2.3 mm² was used in analysis of the roughness of the surfaces. This range allowed for sufficient resolution and representative analysis of the surface.

The image was then processed in the Multi File Analyzer Software to account for any tilt by performing a tilt plane correction. After the image was preprocessed, Rz values were taken on 21-line scans, spaced approximately 30 microns apart, in both x and y directions for 42-line scans total. The Rz values for these 42 scans were averaged for the value referenced herein. The Multi File Analyzer software automatically performs the calculations of Rz with no cutoff wavelengths specified.

### EXAMPLES

### Example 1

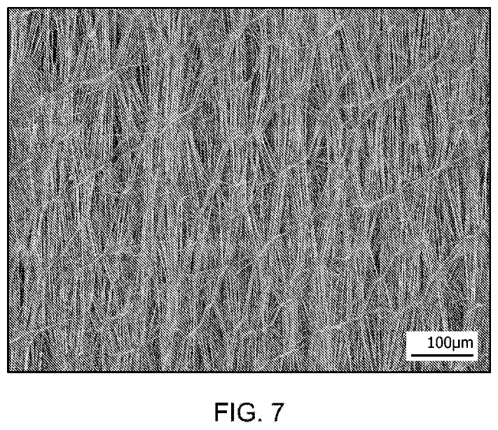

In a first example, a cell encapsulation device having an inactive region with an anchor region thereon was formed. First, a tri-layer membrane of expanded polytetrafluoroethylene (ePTFE) was constructed according to teachings set forth in Example 4 of WO 2020/243668 to Bruhn, et al. The tri-layer ePTFE membrane consisted of a first, tight layer having a plurality of pores with a tight microstructure, a second, open layer having a plurality of pores with an open microstructure, and a third, open layer having a plurality of pores with the most open microstructure. FIG. 7 shows a top-down view of the third, most open ePTFE layer, taken with optical microscope at 50x magnification. Properties of the ePTFE tri-layer are shown in Table 1 below:

TABLE 1
Example 1 ePTFE properties as described in WO2020/243668

| | First, Tight | Second, Open | Third, Most |
|---|---|---|---|
| Description | Layer | Layer | Open Layer |
| Max Pore Size (microns) | 0.5754 | Undeterminable | N/A |
| Pore Size (microns) | 0.987 | Undeterminable | 7.531 |
| Thickness (microns) | 5.45 | 22.6 | 93.12 |

The tri-layer ePTFE membrane was lightly bonded to a sheet of polycarbonate polyurethane film that was approximately 75 microns thick. The bonding was conducted at 165° C. for 30 seconds with a small spring-loaded hand press. This bonding process lightly tacked the polycarbonate polyurethane film to the first tight layer of the tri-layer ePTFE membrane to form an ePTFE/polycarbonate polyurethane composite for handling purposes. The ePTFE/polycarbonate polyurethane composite was then laser cut into the perimeter weld shape of the cell encapsulation device as shown in WO 2020/243668 to Bruhn, et al., component 1340 shown in FIG. 13 therein.

A cell encapsulation device was then formed according to the teachings set forth in WO 2020/243668 to Bruhn, et al., using the method described for Device B. The cell encapsulation devices described herein differ from Device B described in WO 202/243668 to Bruhn, et al. in that (1) no vascularization layer was used and (2) the laser cut ePTFE/polycarbonate polyurethane composite weld layer was substituted for the outermost upper and lower weld film layers described as 1340 in FIG. 13 of WO 202/243668 to Bruhn, et al. The substitution of this ePTFE/polycarbonate polyurethane composite therefore presented an open anchoring region 24 deposited on the upper and lower inactive regions of the encapsulation device.

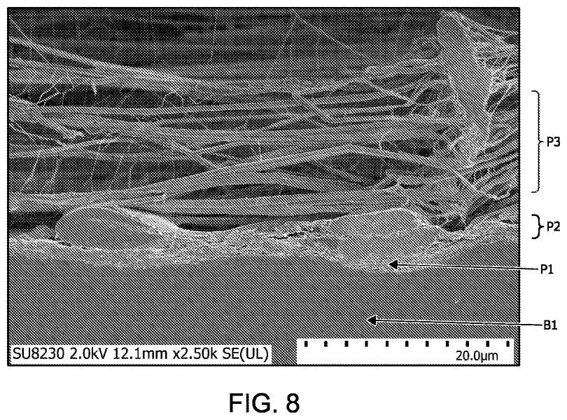

A cross section of the resulting inactive region of the cell encapsulation device is shown in the scanning electron micrograph (SEM) depicted in FIG. 8. More specifically, the polycarbonate polyurethane layer B1 is shown at the bottom of the device penetrating into the first, tight layer of ePTFE P1 to bond the ePTFE composite to the surface of the inactive region (e.g., weld region). The second open ePTFE layer P2 is shown positioned above the layer P1, and the third open ePTFE layer P3 is shown positioned above the layer P2. As illustrated, the second and third open layers P2, P3 retain their open microstructure, allowing for tissue ingrowth and device anchoring.

An optical surface image was taken using Keyence VK-X1000 Laser Scanning Confocal Microscope. Fibril diameter measurements were taken using the and associated Multi File Analyzer Software. Fibril diameters of the finished device in the inactive region were measured, ranging from 0.285 microns to 2.980 microns.

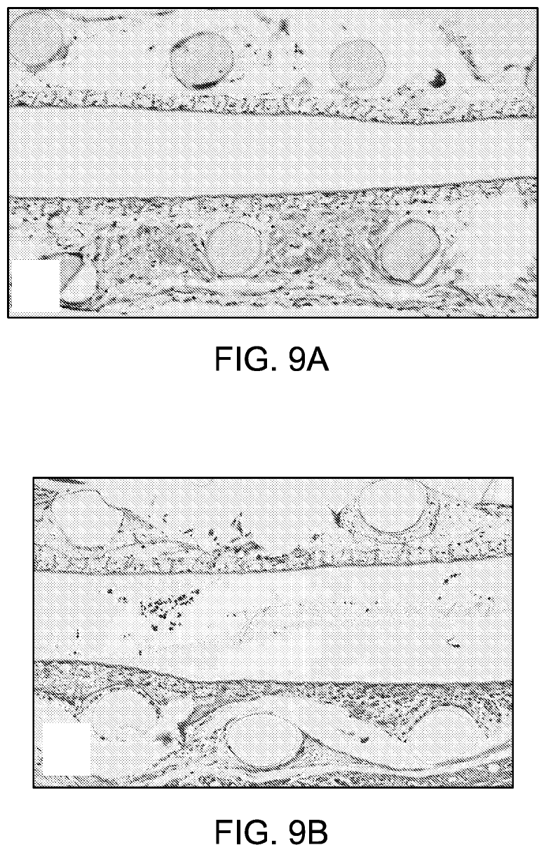

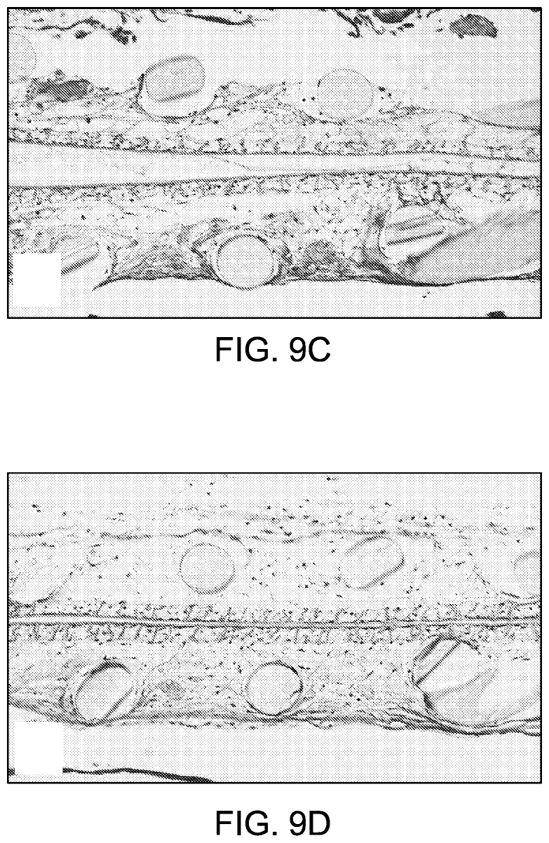

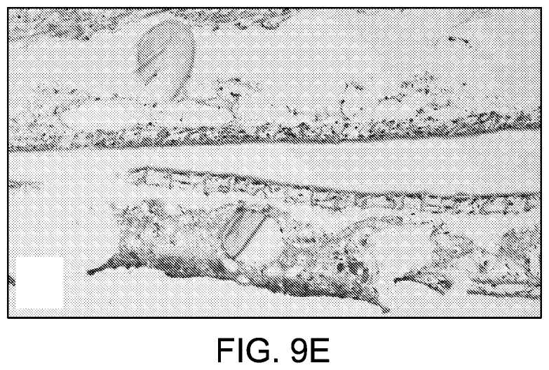

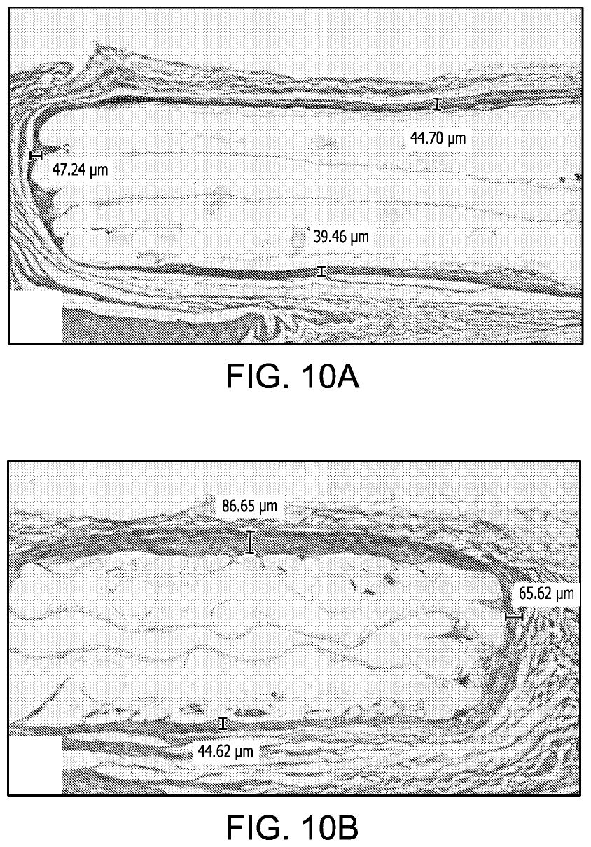

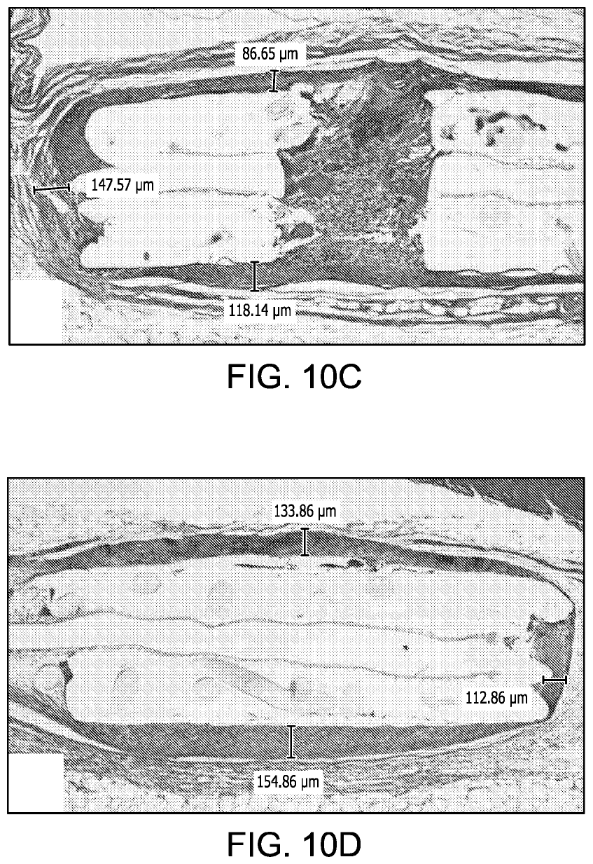

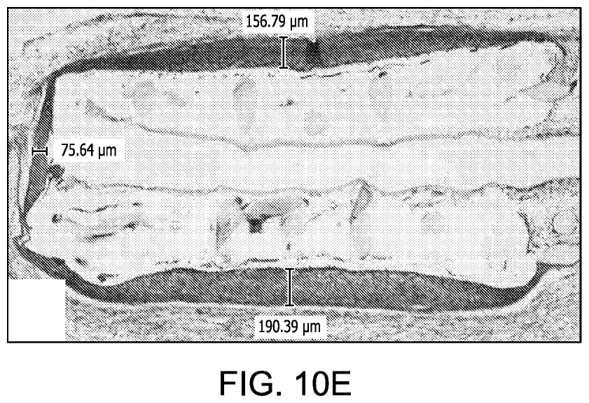

Example 1 devices (i.e., including an anchoring layer around the perimeter of the device) were implanted into New Zealand white rabbits for in vivo evaluation in comparison to Control Devices, described herein as Comparative Example 6 (FIG. 9E). Histological review was performed using H&E staining (i.e., histological staining with both hematoxylin and eosin stains) as well as Trichrome staining. At 7 days post-implant, Example 1 devices showed improved ingrowth into the exposed microstructure of the inactive region relative to the Control Device (shown in FIG. 9E). Additionally, at 7 days post-implant, Example 1 devices demonstrated more extensive and mature collagenized ingrowth into the active areas of the devices (FIG. 9A), relative to the Control Device. Additionally, inactive area capsule thickness was measured via histology. At 30 days, the capsule thickness for Example 1 devices (see FIG. 10A) was lower than that of the Control Device (FIG. 10E), demonstrating improved integration and reduced micromotion and reduced inflammation.

### Example 2

In a second example, a cell encapsulation device having a inactive region with an anchor region was formed. A bilayer membrane of expanded PTFE was constructed according to teachings set forth in Example 2 of WO 2020/243663 to Bruhn, et al. The bilayer membrane consisted of a first tight layer having a plurality of pores with a small pore size, and a second open layer having a plurality of pores with a larger pore size. Properties of the bilayer ePTFE as described in WO 2020/243668 to Bruhn, et al. are shown in Table 2.

TABLE 2
Properties of ePTFE In Example of WO2020/243668

| Description | First, Tight Layer | Second, Open Layer |
|---|---|---|
| Max Pore Size (microns) | 0.3784 | N/A |
| Pore Size (microns) | 0.413 | 4.891 |
| Thickness (microns) | 5.85 | 36.00 |

The bilayer membrane was lightly bonded to a sheet of polycarbonate polyurethane film that is approximately 75 microns thick. The bonding was done at 165° C. for 30 seconds with a small spring-loaded hand press. This bonding process lightly tacked the polycarbonate polyurethane film to the first tight layer of the bilayer ePTFE membrane to form an ePTFE/polycarbonate polyurethane composite for handling purposes. The ePTFE/polycarbonate polyurethane composite was then laser cut into the perimeter weld shape of the cell encapsulation device as shown in WO 2020/243668 to Bruhn, et al., component 1340 shown in FIG. 13 therein.

A cell encapsulation device was then formed according to the teachings set forth in WO 2020/243668 to Bruhn, et al., using the method described for Device B. The cell encapsulation devices in Example 2 differ from WO 202/243668 Device B in that (1) no vascularization layer was used and (2) the laser cut ePTFE/polycarbonate polyurethane composite weld layer was substituted for the outermost upper and lower weld film layers described as 1340 in FIG. 13 of WO 202/243668 to Bruhn, et al. The substitution of this ePTFE/polycarbonate polyurethane composite therefore presented an open anchoring region deposited on the upper and lower inactive regions of the cell encapsulation device.

### Example 3

In a third example, cell encapsulation devices having an inactive region with an anchor region were formed. A cell encapsulation device was formed according to teachings set forth in WO 2020/243668 to Bruhn, et al., using the method described for Device B. The cell encapsulation devices in Example 3 differ from the cell encapsulation devices described in WO 2020/243668 Device B in that (1) an approximately 50 micron thick low density polyethylene (LDPE) film was used in place of the polycarbonate polyurethane weld film layers described as 1340 in FIG. 13 of WO 2020/243668 to Bruhn, et al., (2) no vascularization layer was used, and (3) an ethylene tetrafluoroethylene (ETFE) mesh with nominally 152 micron monofilaments at nominally 250 micron spacing was used. These devices of this Example were processed at a temperature of 160° C.

A non-woven spunbond polyester with a weight of 1.00 oz/yd², a trilobal fiber diameter of nominally 21 microns, and a thickness of 229 micrometers was then incorporated by thermal pressing at 130° C. onto the outermost upper and lower low-density polyethylene (LDPE) weld film layer outer inactive regions of a macroencapsulation device. Example 3 devices showed improved ingrowth into the exposed microstructure of the inactive region (shown in FIG. 9B) relative to the Control Device (shown in FIG. 9E). Capsule thickness for Example 3 devices (see FIG. 10B) was lower than that of the Control Device (FIG. 10E), demonstrating improved integration and reduced micromotion and reduced inflammation.

### Example 4

### Comparative Example

Cell encapsulation devices were formed according to the teachings set forth in WO 2020/243668 to Bruhn, et al., using the method described for Device B. The cell encapsulation devices in Example 4 differ from Device B of WO 202/243668 to Bruhn, et al. in that that no vascularization layer was used, and the outer weld surfaces were perforated as described herein.

After devices were formed, macroscopic holes were punched through the weld region, using a 0.75 mm biopsy punch. Nine holes were punched around the perimeter of the device, through the inactive region, spaced approximately 4.5 mm apart.

Example 4 devices were implanted into New Zealand white rabbits for in vivo evaluation in comparison to Example 6 Control Device through which no holes were punched. In histological review, via H&E (stained with hematoxylin and eosin) and tri-chrome staining 7 days post-implant, Example 4 devices showed no improvement relative to the Control device (see FIG. 9E). No improvement to collagenized tissue ingrowth was seen in the active area of the device, as shown in FIG. 9C. At 28 days, Example 4 device demonstrated a delayed healing response in the area of the punches compared to the rest of the device, as demonstrated by the lack of collagenized ingrowth in the puncture locations. Rather than accelerating the attachment of the device, the large hole required extended time to allow for collagenized tissue integration, providing no noticeable benefit. Capsule thickness for Example 4 devices is shown in FIG. 10C

### Example 5

### Comparative Example

A cell encapsulation device was formed according to the teachings set forth in WO 2020/243668 to Bruhn, et al., using the method described for Device B. The cell encapsulation devices in Example 5 differ from Device B of WO 202/243668 to Bruhn, et al. in that that no vascularization layer was used, and the outer weld surfaces were roughened as described herein. After sealing, microscopic, non-porous features were pressed onto the weld region, using 350 grit sandpaper at 150° C. Non-porous features were about 35 microns in height as measured by confocal laser microscopy on a Keyence VK-X1000 Laser Scanning Confocal Microscope.

Example 5 devices were implanted into New Zealand white rabbits for in vivo evaluation in comparison to Example 6 Control Device (FIG. 9E) to which no surface roughness was added. In histological review, via H&E (stained with hematoxylin and eosin) and tri-chrome staining at 7 and 28 days post-implant, Example 5 devices (FIG. 9D) showed no improvement relative to the control (FIG. 9E). No improvement to collagenized tissue ingrowth was seen in the active area of the device, as demonstrated in FIG. 9D. Capsule thickness for Example 4 devices and control devices are shown in FIG. 10D and FIG. 10E, respectively.

### Example 6

### Comparative Example

A cell encapsulation device having an inactive region with a smooth, nonporous microstructure was formed (FIG. 9E). Example 6 devices were built according to the teaching set forth in WO 2020/243668 to Bruhn, et al., using the method described for Device B. Capsule thickness is shown in FIG. 10E. The Example 6 device differs from Device B of WO 2020/243668 to Bruhn, et al. in that no vascularization layer was used.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A cell encapsulation device, comprising:
a cell retaining region;
an inactive region; and
an anchor region disposed onto at least a portion of the inactive region,
wherein the anchor region contains an open microstructure configured to promote tissue ingrowth.

2. The cell encapsulation device of claim 1, wherein the open microstructure comprises a porous polymer having a plurality of anchoring features defined by a dimension between about 0.1 microns and about 100 microns.

3. The cell encapsulation device of claim 1, wherein the inactive region is arranged along a perimeter of the cell retaining region.

4. The cell encapsulation device of claim 1, comprising at least one inactive region positioned on the cell retaining region.

5. The cell encapsulation device of claim 1, wherein the anchor region comprises expanded polytetrafluoroethylene.

6. The cell encapsulation device of claim 1, wherein at least 15% of an area of the inactive region is covered by the anchor region.

7. The cell encapsulation device of claim 1, wherein an anchor region comprises a bonding layer and an ingrowth layer.

8. The cell encapsulation device of claim 7, wherein the bonding layer comprises a first plurality of fibrils and the ingrowth layer comprises a second plurality of fibrils, and
wherein the bonding layer has a first fibril density that is greater than a second fibril density of the ingrowth layer.

9. The cell encapsulation device of claim 7, wherein the ingrowth layer is configured for permitting tissue ingrowth within the pores of the ingrowth layer.

10. The cell encapsulation device of claim 1, wherein a ratio of active region/inactive region is from 50% to 150%.

11. A cell encapsulation device, comprising:
an active area covering at least one reservoir containing cells and defined by a perimeter and a surface area;
an inactive region arranged around the perimeter of the active region; and
an open microstructure layer disposed onto at least a portion of the anchor region, wherein the open microstructure is defined by interconnected fibrils; and
wherein the open microstructure is defined by a thickness of between 5 microns and 600 microns.

12. The cell encapsulation device of claim 11, wherein the fibrils of the open microstructure have a dimension of between 0.1 microns and 100 microns.

13. The cell encapsulation device of claim 11, wherein the fibrils of the open microstructure have a dimension of less than 1 micron.

14. The cell encapsulation device of claim 11, wherein at least 15% of the inactive region is covered by the anchor region.

15. The cell encapsulation device of claim 11, wherein between 40% and 95% of the inactive region is covered by the anchor region.

16. The cell encapsulation device of claim 11, wherein between 60% and 99% of the inactive region is covered by the anchor region.

17. The cell encapsulation device of claim 11, wherein the open microstructure is welded with the inactive region.

18. The cell encapsulation device of claim 11, wherein the open microstructure is composed of a first layer and a second layer, wherein each of the first layer and the second layer comprise fibrils, and wherein the first layer is defined by a higher first fibril density than a second fibril density of the second layer.

19. An anchor region for use with an implantable medical device configured for facilitating tissue integration, the anchor region comprising:
a bonding layer; and
an ingrowth layer attached to the bonding layer,
wherein the ingrowth layer comprises a porous polymer having a plurality of anchoring features defined by a dimension between about 0.1 microns and about 100 microns; and
wherein the porous polymer is configured for integration with tissue.

20. The anchor region of claim 19, wherein the bonding layer is configured for welding with the implantable medical device.

21. The anchor region of claim 19, wherein the bonding layer has a first plurality of fibrils, the ingrowth layer has a second plurality of fibrils, and wherein a first fibril density of the bonding layer is greater than a second fibril density of the ingrowth layer.
